(19) United States
(12) Patent Application Publication
(10) Pub. No.: US 2010/0021893 A1
(43) Pub. Date: Jan. 28, 2010

(54) Modulators of lanosterol synthetase for treating acne or hyperseborrhea

(75) Inventors: Fernand Labrie, Quebec (CA); Mohamed El-Alfy, Quebec (CA); Van Luu-The, Charny (CA); Ezequiel L. Calvo, Quebec (CA); Jerome Aubert, Grasse (FR); Pascal Collette, Le Cannet (FR); Johannes Voegel, Chateauneuf/Grasse (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/320,171

(22) Filed: Jan. 21, 2009

Related U.S. Application Data
(63) Continuation of application No. PCT/FR2007/051678, filed on Jul. 18, 2007.

(30) Foreign Application Priority Data
Jul. 19, 2006 (FR) 0653026

(51) Int. Cl. C12Q 1/68; C12Q 1/00

(52) U.S. Cl. 435/6; 435/4

(57) ABSTRACT

An in vitro method for screening candidate compounds for the preventive or curative treatment of acne, includes the determination of the capacity of a compound to modulate the expression or the activity of lanosterol synthetase (LSS), and the use of modulators of the expression or activity of this enzyme for the treatment of acne or skin disorders associated with a hyperseborrhea; methods for the in vitro diagnosis or prognosis of these pathologies are also described.

### CROSS-REFERENCE TO PRIORITY/PCT APPLICATIONS

This application claims priority under 35 U.S.C. §119 of FR 0653026, filed Jul. 19, 2006, and is a continuation/national phase of PCT/FR 2007/051678, filed Jul. 18, 2007, and designating the United States (published in the French language on Jan. 24, 2008 as WO 2008/009852 A2; the title and abstract were also published in English), each hereby expressly incorporated by reference in its entirety and each assigned to the assignee hereof.

### BACKGROUND OF THE INVENTION

1. Technical Field of the Invention

The present invention relates to the identification and administration of lanosterol synthetase (LSS) modulating compounds for the treatment of acne and skin disorders associated with a hyperseborrhea. This invention also relates to methods for the in vitro diagnosis or in vitro prognosis of these pathologies.

2. Description of Background and/or Related and/or Prior Art

A hyperseborrheic greasy skin is characterized by excessive secretion and excretion of sebum. Conventionally, a sebum level greater than 200 μg/cm², measured in the region of the forehead, is considered as being characteristic of a greasy skin. A greasy skin is often associated with a desquamation defect, a glistening complexion and a thick skin grain. In addition to these aesthetic disorders, excess sebum can serve as a support for the anarchical development of saprophytic bacterial flora (P. acnes in particular), and cause the appearance of comedones and/or acne lesions.

This stimulation of the production of sebaceous glands is induced by androgens. Acne is in fact a chronic disease of the pilosebaceous follicle under hormonal control. A hormone therapy against acne is one possibility of treatment for women, the aim being to prevent the effects of androgens on the sebaceous gland. In this context, administration is generally made of oestrogens, anti-androgens or agents reducing the production of androgens by the ovaries or the adrenal gland. The anti-androgens administered for the treatment of acne include in particular spironolactone, cyproterone acetate and flutamide. However, these agents have potentially severe side effects. Thus, any pregnancy must be absolutely prevented, in particular because of a risk of feminization for the male foetus. These agents are banned in male patients.

Need therefore exists to identify mediators downstream of the action of the steroid hormones and to modulate them in order to provide a similar therapeutic profile, but with reduced side effects.

### SUMMARY OF THE INVENTION

It has now been discovered that the gene encoding lanosterol synthetase (LSS) was expressed in the human sebaceous glands, and that its expression was regulated by androgens, in vivo, in a mouse preputial gland model. Thus, targeting the LSS gene or its expression product is now proposed to prevent and/or improve acne and skin disorders associated with a hyperseborrhea, in particular the appearance of greasy skin.

The expression acne means all the forms of acne, namely, in particular acne vulgaris, comedo type acne, polymorphic acne, nodulocystic acne, acne conglobata, or secondary acnes such as solar acne, acne medicamentosa or occupational acne. This invention also provides in vitro diagnostic or in vitro prognostic methods based on the detection of the level of expression or activity of LSS.

### BRIEF DESCRIPTION OF THE DRAWINGS

FIGS. 1A and 1B are graphs which show the measurement of the expression of the LSS gene in gonadectomized male mice treated with various vehicles,

FIGS. 2A and 2B are graphs presenting a kinetic study of 15 minutes to 96 hours,

FIGS. 3A, 3B and 3C show the expression of LSS in the sebaceous gland of the mouse skin by in situ hybridization, and

FIGS. 4A, 4B and 4C show the expression of LSS in the preputial gland of mice by in situ hybridization.

### DETAILED DESCRIPTION OF BEST MODE AND SPECIFIC/PREFERRED EMBODIMENTS OF THE INVENTION

### LSS

The enzyme LSS denotes lanosterol synthetase, also called 2,3-epoxysqualene-lanosterol cyclase, 2,3-oxidosqualene-lanosterol cyclase, OSC, or oxidosqualene-lanosterol cyclase.

Lanosterol synthetase catalyzes the cyclization of (S)-2,3-oxidosqualene to lanosterol during the reaction which forms the sterol nucleus. The gene encoding lanosterol synthetase is called OSC gene or, in the context of the present invention, LSS gene. The LSS gene is considered as an important target for the treatment of hypercholesterolemia (Huff and Telford, 2005, Trends Pharmacol Sci., 2005 July; 26(7): 335-40) but also for the treatment of Chagas disease (Hankins, Gillesprie, Aikenhead and Buckner, 2005, Mol Biochm Parasitol., 2005 November; 144(1): 68-75). The inhibition of lanosterol synthetase makes it possible to reduce the production of lanosterol and therefore the synthesis of cholesterol. It also causes an increase in the synthesis of oxysterols. This dual action makes it possible to amplify the reduction in cholesterol during a treatment (Telford et al., 2005, Arteriocler Thromb Vasc Biol., 2005 December; 25(12): 2608-14). The inhibitors involved are many and were produced from extracts of fungi (Sakano, Shibuya, Yamaguchi, Masuma, Tomoda, Omura, Ebizuka, 2004, J. Antibiot., 2004 September; 57(9): 564-8), plants, or derived from the endogenous ligand and sugars such as mono- and digalactosides (Tanaka, Sakano, Nagatsu, Shibuya, Ebizuka, Goda, 2005, Bioorg Med Chem. Lett., 2005 Jan. 3; 15(1): 159-62) or the inhibitor Ro 48-8071 cited in the publications: Morand, Aebi, Dehmlow, Ji, Gains, Lengsfeld, Himber, 1997 J Lipid Red 38, 373-390 and Thoma et al., 2004 Nature November 4; 432, 118-122.

In the context of the invention, the term “LSS gene” or “LSS nucleic acid” means the gene or nucleic acid sequence which encodes lanosterol synthetase. If the intended target is preferably the human gene or its expression product, the invention may also call into play cells expressing a heterologous lanosterol synthetase, through genomic integration or transient expression of an exogenous nucleic acid encoding the enzyme.

A human cDNA sequence for LSS is reproduced in the annex (SEQ ID No. 1). It is the sequence NM_{—}002340 whose coding part is located from nucleic acid 33 to 2231.

Diagnostic Applications:

The present invention features an in vitro method for the diagnosis or monitoring of the progression of acne lesions or of a skin disorder associated with a hyperseborrhea in a subject, comprising comparing the expression or activity of the protein lanosterol synthetase (LSS), the expression of its gene or the activity of at least one of its promoters, in a biological sample from a subject compared with a biological sample from a control subject.

The expression of the protein may be determined by an assay of the LSS protein by radioimmunoassay, for example by ELISA assay. Another method, in particular for measuring the expression of the LSS gene, is to measure the quantity of corresponding mRNA, by any method as described above. An assay of the activity of LSS may also be employed.

In the context of a diagnosis, the “control” subject is a “healthy” subject.

In the context of a monitoring of the progression of acne lesions or of a skin disorder linked to a hyperseborrhea, the “control subject” refers to the same subject at a different time, which preferably corresponds to the start of the treatment (To). This measurement of the difference in the expression or activity of LSS, or the expression of its gene or the activity of at least one of its promoters, makes it possible in particular to monitor the efficacy of a treatment, in particular a treatment with an LSS modulator, as indicated above, or another treatment against acne or a skin disorder associated with a hyperseborrhea. Such a monitoring can reassure the patient regarding the justification or the need for pursuing this treatment.

The present invention also features an in vitro method for determining the predisposition of a subject to develop acne lesions or a skin disorder associated with a hyperseborrhea, comprising comparing the expression or the activity of the protein lanosterol synthetase (LSS), the expression of its gene or the activity of at least one of its promoters, in a biological sample from a subject compared with a biological sample from a control subject.

Here again, the expression of the LSS protein may be determined by an assay of this protein by radioimmunoassay, for example by ELISA assay. Another method, in particular for measuring the expression of the LSS gene, is to measure the quantity of corresponding mRNA by any method as described above. An assay of the activity of LSS may also be employed.

The subject tested is here an asymptomatic subject with no skin disorder linked to a hyperseborrhea or an acne. The “control” subject in this method means a “healthy” reference subject or population. The detection of this predisposition allows the putting in place of a preventive treatment and/or an increased monitoring of the signs linked to acne or to a skin disorder associated with a hyperseborrhea.

In these in vitro diagnostic or prognostic methods, the biological test sample may be any biological fluid sample or a sample of a biopsy. Preferably, the sample may be a preparation of skin cells obtained for example by desquamation or biopsy. It may also be sebum.

Screening Methods:

This invention also features an in vitro method for screening candidate compounds for the preventive and/or curative treatment of acne, or any skin disorder associated with a hyperseborrhea, comprising determining the capacity of a compound to modulate the expression or activity of lanosterol synthetase or the expression of its gene or the activity of at least one of its promoters, the said modulation indicating the usefulness of the compound for the preventive or curative treatment of acne or any skin disorder associated with a hyperseborrhea. The method therefore makes it possible to select the compounds capable of modulating the expression or activity of LSS, or the expression of its gene or the activity of at least one of its promoters.

More particularly, this invention features an in vitro method for screening candidate compounds for the preventive and/or curative treatment of acne or skin disorders associated with a hyperseborrhea, comprising the following steps:

a. preparing at least two biological samples or reaction mixtures;

b. bringing one of the samples or reaction mixtures into contact with one or more test compounds;

c. measuring the expression or activity of the protein lanosterol synthetase, the expression of its gene or the activity of at least one of its promoters, in biological samples or reaction mixtures;

d. selecting the compounds for which a modulation of the expression or activity of the protein lanosterol synthetase, the expression of its gene or the activity of at least one of its promoters, is measured in the sample or mixture treated in b), compared with the untreated sample or mixture.

The expression “modulation” means any effect on the expression or activity of the enzyme, the expression of the gene or the activity of at least one of its promoters, namely, optionally a partial or complete stimulation, but preferably a partial or complete inhibition. Thus, the compounds tested in step d) above preferably inhibit the expression or activity of the protein lanosterol synthetase, the expression of its gene or the activity of at least one of its promoters. The difference in expression obtained with the test compound compared with a control prepared in the absence of the compound is significant from 25% or more.

In the present text, unless otherwise specified, “expression of a protein” means the quantity of this protein.

The expression “activity of a protein” means its biological activity.

The expression “activity of a promoter” means the capacity of this promoter to trigger the transcription of the DNA sequence coded downstream of this promoter (and therefore indirectly the synthesis of the corresponding protein).

The test compounds may be of any type. They may be of a natural origin or may have been produced by chemical synthesis. This may be a library of structurally defined chemical compounds, non-characterized compounds or substances or a mixture of compounds.

Various techniques may be used to test these compounds and identify the compounds of therapeutic interest, modulators of the expression or the activity of lanosterol synthetase.

According to a first embodiment, the biological samples are cells transfected with a reporter gene that is operably linked to all or part of the promoter of the gene encoding lanosterol synthetase, and step c) described above entails measuring the expression of the said reporter gene.

The reporter gene may in particular encode an enzyme which, in the presence of a given substrate, leads to the formation of colored products, such as CAT (chloramphenicol acetyltransferase), GAL (beta-galactosidase) or GUS (beta-glucuronidase). This may also be the luciferase gene or GFP (Green Fluorescent Protein). The assay of the protein encoded by the reporter gene, or its activity, is carried out in a conventional manner by calorimetric, fluorometric or chemiluminescent techniques, among others.

According to a second embodiment, the biological samples are cells expressing the gene encoding lanosterol synthetase, and step c) described above entails measuring the expression of the said gene.

The cell employed here may be of any type. This may be a cell endogenously expressing the LSS gene, such as, for example, a liver cell, an ovarian cell or even better a sebocyte. It is also possible to employ organs of human or animal origin, such as for example the preputial gland, clitorial gland or sebaceous gland of the skin.

This may also be a cell transformed with a heterologous nucleic acid encoding lanosterol synthetase, preferably of human origin, or of mammalian origin.

A wide variety of host cell systems may be employed, such as, for example, Cos-7, CHO, BHK, 3T3, HEK293 cells. The nucleic acid may be stably or transiently transfected by any method known to one skilled in this art, for example using calcium phosphate, DEAE-dextran, liposome, viruses, electroporation or microinjection.

In these methods, the expression of the LSS gene or of the reporter gene may be determined by evaluating the level of transcription of the said gene, or its level of translation.

The expression level of transcription of a gene means the quantity of corresponding mRNA produced.

The expression level of translation of a gene means the quantity of protein produced.

One skilled in this art is familiar with techniques allowing the quantitative or semi-quantitative detection of the mRNA of a gene of interest. The techniques based on the hybridization of mRNA with specific nucleotide probes are the most common (Northern Blot, RT-PCR, protection using RNase). It may be advantageous to employ detection markers such as fluorescent, radioactive or enzymatic agents or other ligands (for example avidin/biotin).

In particular, the expression of the gene may be measured by real-time PCR or by protection using RNase. The expression protection using RNase means the detection of a known mRNA among poly(A) RNAs of a tissue, which may be carried out with the aid of a specific hybridization with a labeled probe. The probe is a labeled (radioactive) complementary RNA for the messenger to be detected. It may be constructed from a known mRNA whose cDNA, after RT-PCR, has been cloned into a phage. The poly(A) RNA of the tissue where the sequence is to be detected is incubated with this probe under slow hybridization conditions in liquid medium. RNA:RNA hybrids are formed between the mRNA to be detected and the anti-sense probe. The hybridized medium is then incubated with a mixture of ribonucleases specific for single-stranded RNA, such that only the hybrids formed with the probe can withstand this digestion. The product of digestion is then deproteinized and repurified before being analyzed by electrophoresis. The labeled hybridized RNAs are detected by autoradiography.

The level of translation of the gene is evaluated for example by immunological assay of the product of the said gene. The antibodies employed for this effect may be of the polyclonal or monoclonal type. Their production involves conventional techniques. An anti-lanosterol synthetase polyclonal antibody may, inter alia, be obtained by immunization of an animal such as a rabbit or a mouse, with the whole enzyme. The anti-serum is collected and then depleted according to methods known per se by persons skilled in the art. A monoclonal antibody may, inter alia, be obtained by the conventional Köhler and Milstein method (Nature (London), 256: 495-497 (1975)). Other methods of preparation of monoclonal antibodies are also known. It is possible, for example, to produce monoclonal antibodies by expressing a nucleic acid cloned from a hybridoma. It is also possible to produce antibodies by the phage display technique by introducing antibody cDNAs into vectors, which are typically filamentous phages which display V gene libraries at the surface of the phage (for example, fUSE5 for E. coli).

The immunological assay may be carried out in a solid phase or in a homogeneous phase; in a single stage or in two stages; as a sandwich method or as a competitive method, by way of non-limiting examples. According to a preferred embodiment, the capture antibody is immobilized on a solid phase. It is possible to employ, by way of non-limiting examples of a solid phase, microplates, in particular polystyrene microplates, or solid particles or beads, paramagnetic beads.

ELISA assays, radio-immunoassays or any other detection technique may be carried out in order to reveal the presence of the antigen-antibody complexes formed.

The characterization of the antigen-antibody complexes, and more generally of the isolated or purified proteins, but also recombinant proteins (obtained in vitro and in vivo), may be carried out by mass spectrometry analysis. This identification is made possible by virtue of the analysis (determination of the mass) of peptides generated by the enzymatic hydrolysis of the proteins (trypsin in general). Generally, the proteins are isolated according to methods known to one skilled in this art, prior to the enzymatic digestion. The analysis of the peptides (in hydrolysate form) is performed by separation of the peptides by HPLC (nano-HPLC) based on their physicochemical properties (reversed phase). The determination of the mass of the peptides thus separated is carried out by ionization of the peptides or by direct coupling to mass spectrometry (electrospray ESI mode), or after deposition and crystallization in the presence of a matrix known to one skilled in this art (analysis in MALDI mode). The proteins are then identified using appropriate software (for example Mascot).

According to a third embodiment, step a) described above entails preparing reaction mixtures each comprising an enzyme lanosterol synthetase and a substrate of the enzyme, and step c) described above entails measuring the enzyme activity.

The enzyme lanosterol synthetase may be produced according to customary techniques using Cos-7, CHO, BHK, 3T3 and HEK293 cells. It may also be produced with the aid of microorganisms such as bacteria (for example, E. coli or B. subtilis), yeasts (for example Saccharomyces, Pichia) or insect cells, such as Sf9 or Sf21.

The determination of the enzymatic activity preferably comprises the determination of the synthetase activity, by extraction of the sterols produced and chromatographic analysis.

Assays of the enzymatic activity of LSS are described in the literature (see for example Kusano et al, 1991, Chem. Pharm. Bull., 39, 239-241, or Morand et al, Journals of Lipid Research, 1997, 38: 373-390).

Thus, the activity of lanosterol synthetase may be evaluated in the following manner: the cDNA for lanosterol synthetase is expressed under the control of a glyceraldehyde 3-phosphate dehydrogenase promoter in a yeast deficient in lanosterol synthetase, GIL77 (Kushiro et al., Eur. J. Biochem., 256, 238-241,1998). The acellular extract obtained from the transformed yeast cell is used. For this, the yeast is homogenized in a buffer A [potassium phosphate buffer 0.1M (pH 7.4), sucrose 0.45M, EDTA 1 mM and dithiothreitol 1 mM] in the presence of acid-treated glass beads. The supernatant obtained by centrifugation is brought to the concentration of 10 mg/ml by adding buffer A.

A ¹⁴C(3S)-2,3-oxidosqualene substrate is biosynthetically prepared by culturing lanosterol-deficient GL7 yeast cells (Gollub et al., J. Biol. Chem., 252, 2846-2854, 1977) in the presence of sodium ¹⁴C-acetate in order to incorporate the radioactivity into the oxidosqualene.

The substrate (final concentration, 0.17-0.42 μM, 4.5 nCi) and the test compound are added to the buffer B [0.1M potassium phosphate buffer (pH 7.4) and 0.1% Triton X-100] in order to obtain a total volume of 900 μl, preincubated at 37° C. for 10 minutes, before the addition of the enzyme (1 mg) in order to prepare a solution of 1 ml, incubated at 37° C. for 60 minutes. 6% potassium hydroxide/ethanol is added in order to stop the reaction, and the mixture is saponified by incubation at 37° C., for 10 minutes, before addition of cyclohexane (2 ml). The cyclohexane layer is dried, deposited on the thin-layer chromatography plate and developed with a benzene/acetone solvent (19/1, v/v). The quantities of unreacted substrate and of ¹⁴C-lanosterol are then determined by an analyzer BAS-1500 (Fuji Photo Film Co., Japan), in order to calculate the level of inhibition of the synthesis of lanosterol.

Modulators of the Enzyme:

The present invention also features the use of a modulator of the human enzyme lanosterol synthetase which can be obtained by one of the above methods, for the preparation of a medicament intended for the preventive and/or curative treatment of acne, or of skin disorders associated with a hyperseborrhea.

A method for the preventive and/or curative treatment of acne, or of skin disorders associated with a hyperseborrhea, is thus described here, the regime or regimen comprising the administration of a therapeutically effective quantity of a modulator of the human enzyme lanosterol synthetase, to a patient requiring such a treatment.

This invention also features the cosmetic application of a modulator of the human enzyme lanosterol synthetase for the aesthetic treatment of greasy skins.

Preferably, the modulator is an inhibitor of the enzyme. The term “inhibitor” refers to a chemical compound or substance which substantially eliminates or reduces the enzymatic activity of lanosterol synthetase. The term “substantially” means a reduction of at least 25%, preferably of at least 35%, preferably still of at least 50%, and more preferably of at least 70% or 90%. More particularly, it may be a compound which interacts with, and blocks, the catalytic site of the enzyme, such as compounds of the competitive inhibitor type.

A preferred inhibitor interacts with the enzyme in solution at inhibitor concentrations of less than 1 μM, preferably of less than 0.1 μM, preferably still of less than 0.01 μM.

The modulator compound may be an anti-lanosterol synthetase inhibitory antibody, preferably a monoclonal antibody. Advantageously, such an inhibitory antibody is administered in a quantity sufficient to obtain a plasma concentration of about 0.01 μg per ml to about 100 μg/ml, preferably of about 1 μg per ml to about 5 μg/ml.

The modulator compound may also be a polypeptide, a DNA or RNA anti-sense polynucleotide, an si-RNA or a PNA (“peptide nucleic acid”, polypeptide chain substituted with purine and pyrimidine bases whose spatial structure mimics that of DNA and allows hybridization thereto).

Several lanosterol synthetase inhibitors are known, and are proposed for the treatment of hypercholesterolemia. The invention comprises the administration of such lanosterol synthetase inhibiting compounds for the preventive and/or curative treatment of acne or skin disorders associated with a hyperseborrhea. Without limitation, exemplary is Ro 48-8071 (or [4′-(6-allylmethylaminohexyloxy)-2′-fluorophenyl]-(4-bromophenyl)methanone fumarate) as lanosterol synthetase inhibitor.

Other modulator compounds identified by the screening method described above are also useful.

The modulator compounds are formulated in a pharmaceutical composition, in combination with a pharmaceutically acceptable vehicle. These compositions may be administered for example orally, enterally, parenterally or topically. Preferably, the pharmaceutical composition is applied topically. By the oral route, the pharmaceutical composition may be provided in the form of tablets, gelatin capsules, sugar-coated tablets, syrups, suspensions, solutions, powders, granules, emulsions, suspensions of microspheres or nanospheres or lipid or polymer vesicles allowing controlled release. By the parenteral route, the pharmaceutical composition may be provided in the form of solutions or suspensions for infusion or injection.

By the topical route, the pharmaceutical composition is more particularly useful for the treatment of the skin and the mucous membranes and may be provided in the form of salves, creams, milks, ointments, powders, impregnated pads, solutions, gels, sprays, lotions or suspensions. It may also be provided in the form of suspensions of microspheres or nanospheres or of lipid or polymer vesicles or of polymer patches or hydrogels allowing controlled release. This composition for topical application may be provided in anhydrous form, in aqueous form or in the form of an emulsion. In a preferred embodiment, the pharmaceutical composition is provided in the form of a gel, a cream or a lotion.

The composition may comprise an amount of LSS modulator ranging from 0.001 to 10% by weight, in particular from 0.01 to 5% by weight relative to the total weight of the composition.

The pharmaceutical composition may additionally contain inert additives or combinations of these additives, such as:

wetting agents;

taste enhancing agents;

preservatives such as para-hydroxybenzoic acid esters;

stabilizing agents;

moisture regulating agents;

pH regulating agents;

osmotic pressure modifying agents;

emulsifying agents;

UV-A and UV-B screening agents;

and antioxidants, such as alpha-tocopherol, butylated hydroxyanisole or butylated hydroxytoluene, Super Oxide Dismutase, Ubiquinol or certain metal chelators.

### LEGEND FOR THE FIGURES

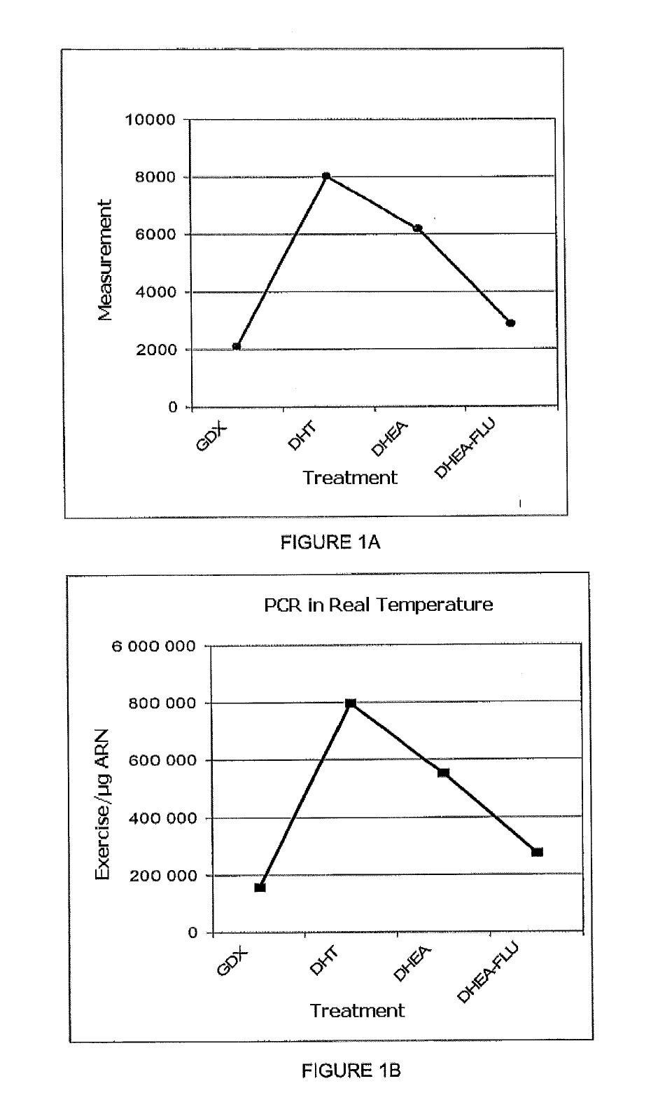

FIGS. 1A and 1B are graphs which show the measurement of the expression of the LSS gene in gonadectomized male mice treated with the vehicle, DHT, DHEA or the combination of DHEA-Flutamide for a period of 7 days once per day (long-term treatment). The results obtained by the Affymetrix technique (FIG. 1A) were confirmed by the real-time RT-PCR technique (FIG. 1B).

GDX: gonadectomized mice treated with the vehicle.

DHT: gonadectomized mice treated with Dihydrotestosterone (agonist of the androgen receptor).

DHEA: gonadectomized mice treated with Dihydroepiandrosterone (precursor of the steroid hormones; in the preputial glands metabolized to the active androgen).

DHEA-Flu: gonadectomized mice treated with a combination of Dihydroepiandrosterone and Flutamide (antagonists of the androgen receptor; which blocks the effects of the DHT and DHEA agonists).

Level of expression: level of expression of the mRNA.

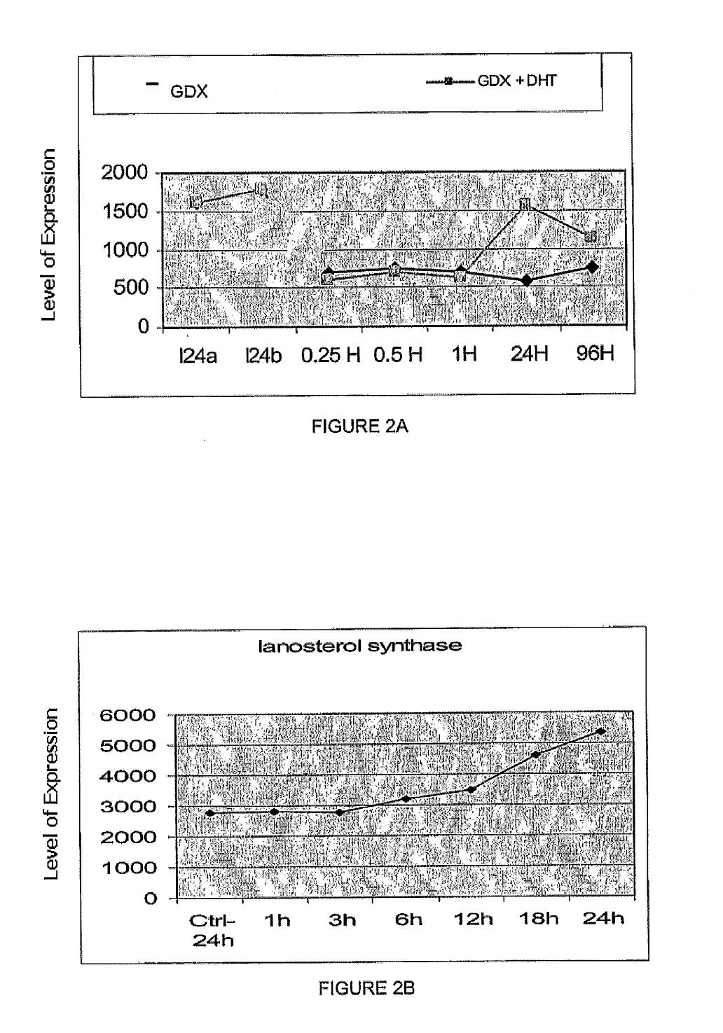

FIGS. 2A and 2B are graphs presenting a kinetic study of 15 minutes to 96 hours (FIG. 2A) and a kinetic study of 1 hour to 24 hours (FIG. 2B). In FIG. 2A, points 124a and 124b show the level of expression of lanosterol synthetase of control mice (=non-gonadectomized mice; duplicate) at the 24 hour point. The next points are from gonadectomized mice and indicate the successive times (in hours) of the kinetic study.

Level of expression: level of expression of mRNA.

Square: expression in the gonadectomized mice following treatment with DHT at the time zero.

Diamond: expression in gonadectomized mice without DHT treatment.

In FIG. 2B, the Ctrl-24 h point shows the level of expression of lanosterol synthetase of gonadectomized mice not treated with DHT at the 24 hour point. The next points are obtained from gonadectomized mice treated with DHT and indicate the successive times (in hours) of the kinetic study.

Level of expression: level of expression of the mRNA.

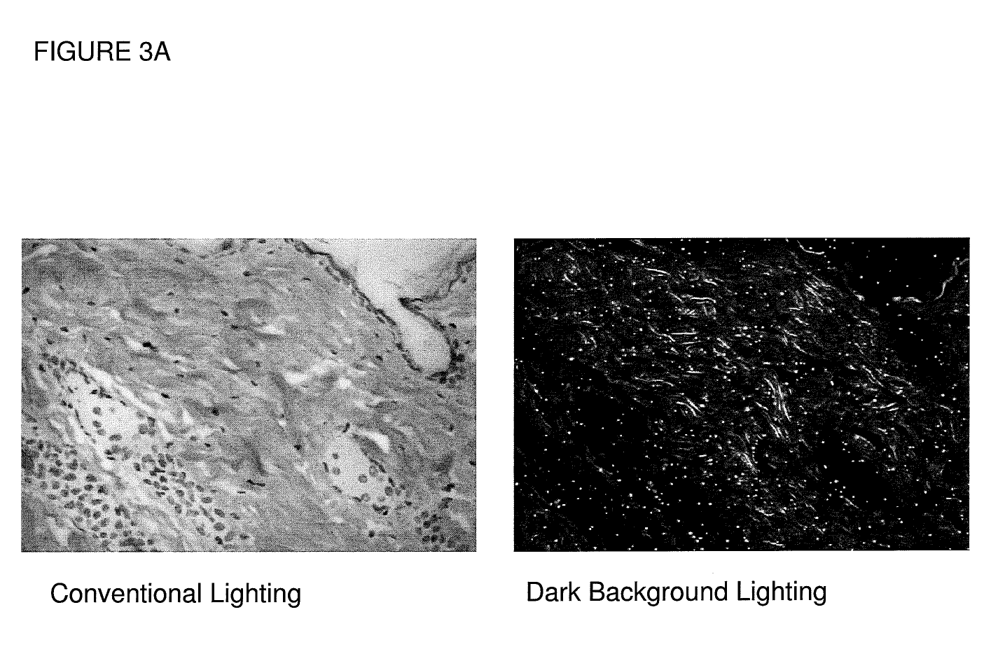

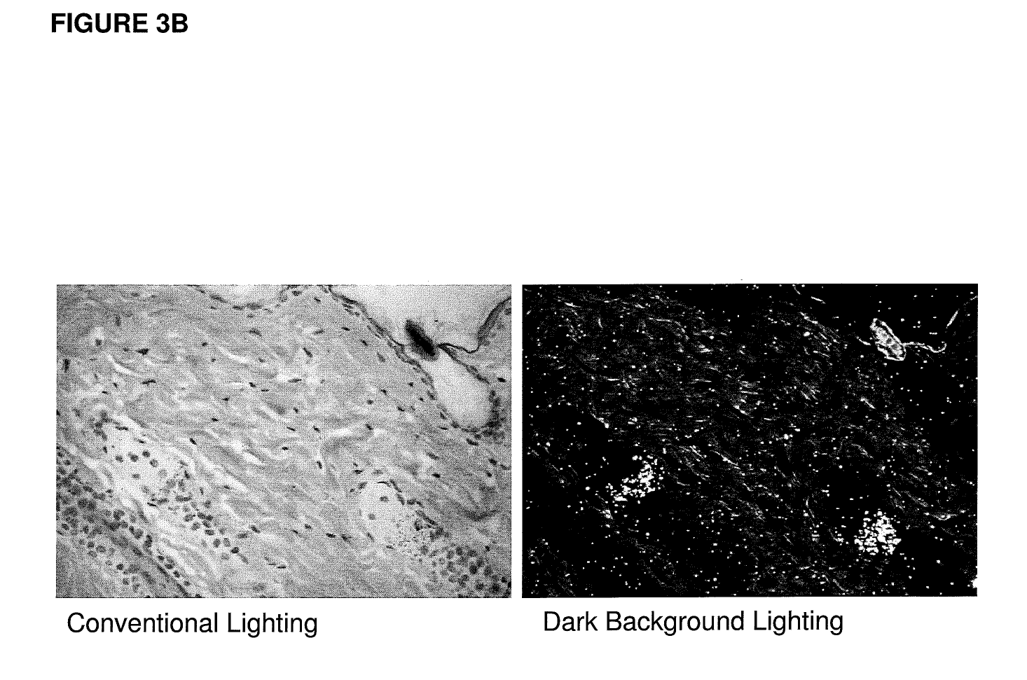

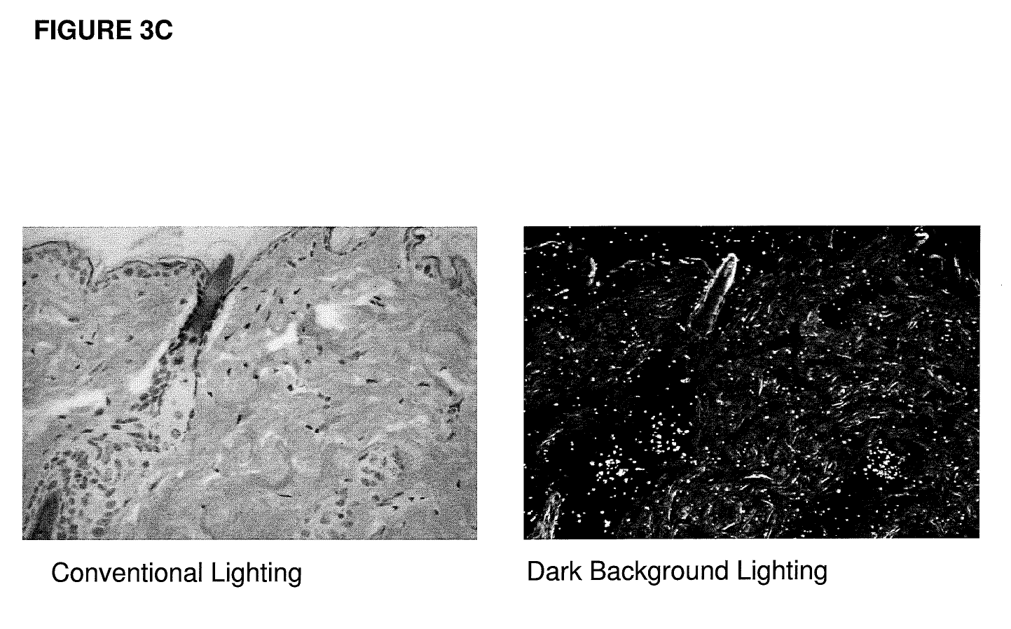

FIGS. 3A, 3B and 3C show the expression of LSS in the sebaceous gland of the mouse skin by in situ hybridization. FIG. 3A is a photograph under conventional illumination and under illumination with a dark background of a mouse skin section subjected to in situ hybridization using an LSS sense probe (negative control; intact animal 44). FIG. 3B is a photograph under conventional illumination and under illumination with a dark background of a mouse skin section subjected to in situ hybridization with an anti-sense probe, in an intact animal (animal 44). FIG. 3C is a photograph under conventional illumination and under illumination with a dark background of a mouse skin section subjected to an in situ hybridization with an anti-sense probe, in a gonadectomized animal (animal 53).

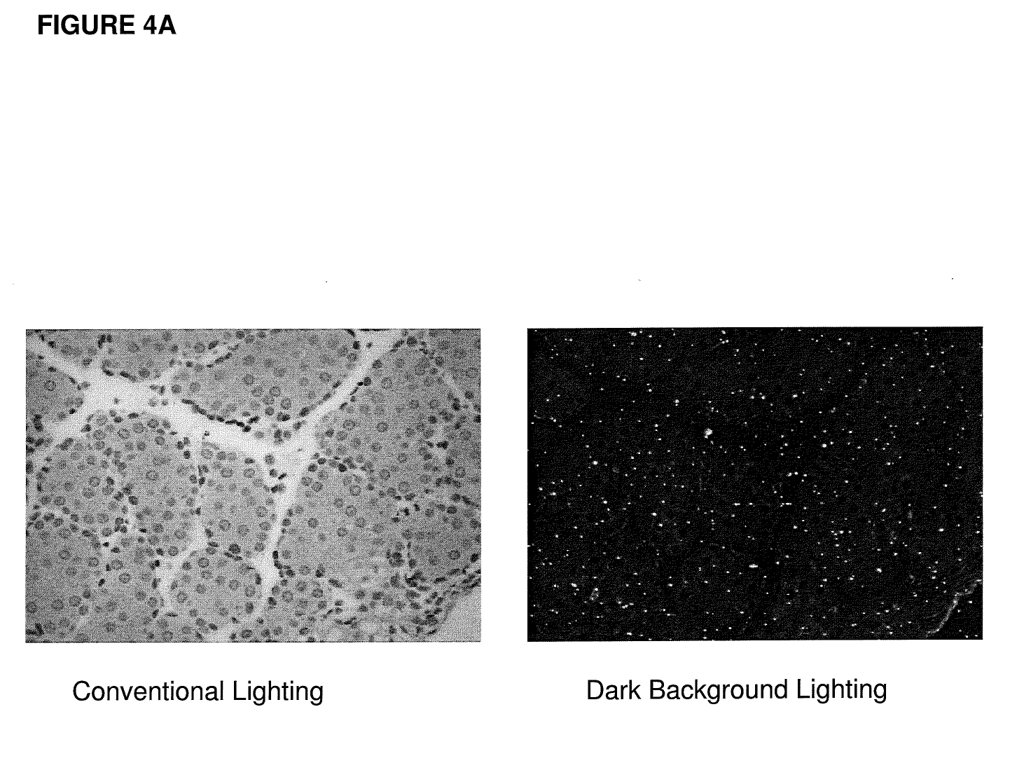

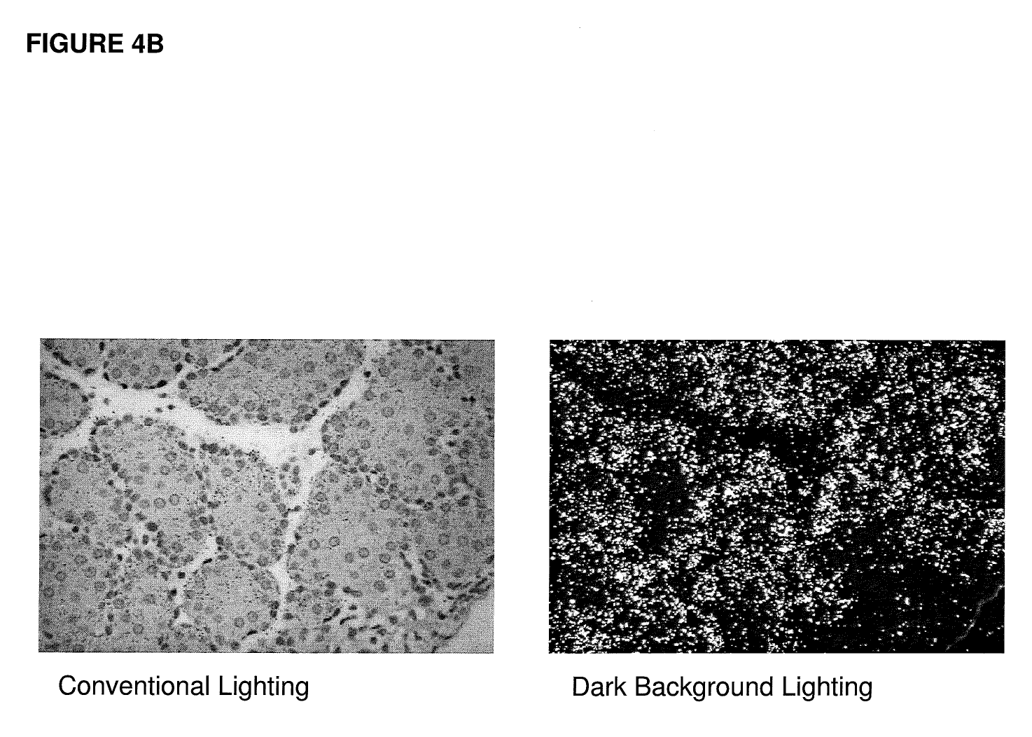

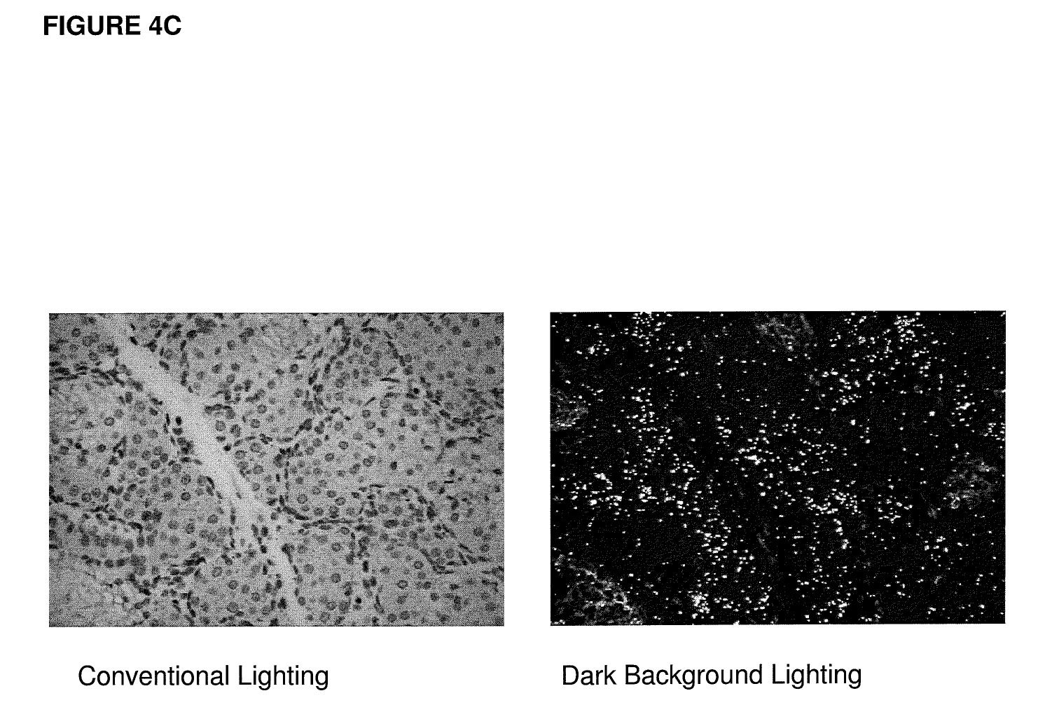

FIGS. 4A, 4B and 4C show the expression of LSS in the preputial gland of mice by in situ hybridization. FIG. 4A is a photograph under conventional illumination and under illumination with a dark background of a mouse prepuce section subjected to an in situ hybridization with an LSS sense probe (negative control; animal 45). FIG. 4B is a photograph under conventional illumination and under illumination with a dark background of a mouse prepuce section subjected to an in situ hybridization with an anti-sense probe, in an intact animal (animal 45).

FIG. 4C is a photograph under conventional illumination and under illumination with a dark background of a mouse prepuce section subjected to an in situ hybridization with an anti-sense probe, in a gonadectomized animal (animal 53).

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Experimental Data

### Example 1

### Expression of Lanosterol Synthetase in the Human Sebaceous Gland and in the Human Epidermis

Human sebaceous glands were separated from the human epidermis by treatment with dispase and dissection under a binocular lens. Samples of total RNA were prepared from the sebaceous glands and from the epidermis.

The expression of the genes was analyzed on an Affymetrix station (microfluidic model; hybridization oven; scanner; computer) following the protocols provided by the company. Briefly, the total RNA isolated from the tissues is transcribed to cDNA. From the double-stranded cDNA, a cRNA labeled with biotin is synthesized using T7 polymerase and a precursor NTP conjugated to biotin. The cRNAs are then fragmented to small sized fragments. All the molecular biology steps are checked using the Agilent “Lab on a chip” system in order to confirm the good efficiencies of the enzymatic reactions. The Affymetrix chip is hybridized with the biotinylated cRNA, rinsed and then fluorescence labeled using a fluorophore conjugated to streptavidin. After washings, the chip is scanned and the results are calculated using the MAS5 software provided by Affymetrix. An expression value is obtained for each gene as well as the indication of the significance of the value obtained. The calculation of the significance of the expression is based on the analysis of the signals, which are obtained following hybridization of the cRNA of a given gene with an oligonucleotide that is a perfect match compared with an oligonucleotide which contains a single mismatch in the central region of the oligonucleotide (see Table 1).

TABLE 1
measurement of the expression of lanosterol synthetase in the epidermis and in
the human sebaceous gland through the use of the Affymetrix chip technology.

| | | | | Significance | Significance |
|---|---|---|---|---|---|
| | | Expression | Expression | of the expression* | of the expression* |
| Affymetrix | Name of | in the human | in the human | in the human | in the human |
| identifier | the gene | sebaceous gland | epidermis | sebaceous gland | epidermis |
| 202245_at | lanosterol synthetase | 302 | 255 | 1 | 1 |
| | (2,3-oxidosqualene- | | | | |
| | lanosterol cyclase) | | | | |

*Indicator of the significance of the expression of the gene analyzed in the sample indicated: presence (=1) or absence (=0).

Results:

Lanosterol synthetase is well expressed in both tissues (sebaceous gland, epidermis). Differential analysis between the expression in the human sebaceous gland and the human epidermis shows that the slightly higher expression in the sebaceous gland is not significant compared with the value observed in the epidermis (Table 1).

### Example 2

### Expression of Lanosterol Synthetase in the Mouse Preputial Gland

A. The mouse preputial glands show differentiation of the sebocyte type and are used as an experimental model for a sebaceous gland. They have a sufficient size to allow isolation of RNA without having recourse to microdissection technologies.

Analysis of the expression of lanosterol synthetase in the mouse preputial glands was carried out under conditions of deficiencies of steroid hormones (in particular of androgenic hormones) following a gonadectomy. The gonadectomized animals were then treated with physiological quantities of Dihydrotestosterone (DHT) or Dihydroepiandrosterone (DHEA) in order to restore a physiological level of androgenic hormones, or as a control experiment with a DHEA-Flutamide combination in which the Flutamide, an antagonist of the androgen receptors, blocks the effect of DHEA. Comparison of the gene expression under these experimental conditions makes it possible to unambiguously identify the modulation or non-modulation of the gene expression of a gene in question by the androgenic hormones.

The gene expression was analyzed using the Affymetrix technology described above (FIG. 1A) and the results were then confirmed by the real-time PCR technique (FIG. 1B).

The real-time PCR was carried out using the protocols provided by the company Applied Biosystems using the 7900HT Sequence Detection System. The total RNA isolated from the tissues is transcribed (RT) to cDNA and the latter is amplified by PCR (Polymerase Chain Reaction). The progress of the PCR is monitored in real time using fluorescent TaqMan probes which allow precise quantification of the quantity of mRNA of a given gene present in the biological sample at the start.

Result:

The mRNA for lanosterol synthetase is induced by a chronic treatment for 7 days with androgens in the preputial gland.

B. Male mice were gonadectomized and were then treated with the vehicle or DHT. The preputial glands were removed for a period ranging up to 4 days (androgenic treatment alone—observation of a short-term kinetics). The RNA was isolated and the expression of the genes was analyzed by the Affymetrix technique. FIG. 2A and FIG. 2B represent the relative level of expression of the mRNA as a function of time.

Results:

Gonadectomy (which causes a steroid hormone deficiency) induces a reduction in the quantity of mRNA for lanosterol synthetase in the mouse preputial gland.

The mRNA for lanosterol synthetase in the mouse preputial gland is induced by a short-term treatment with DHT (effect visible at 18, 24 and 96 hours).

### Example 3

### Expression of LSS in the Sebaceous Gland of Mouse Skin by “In Situ Hybridization”

Methods:

Sense and anti-sense probes were prepared from the LSS gene by incubation of the linearized gene (2 μg) with 63 μCi of [³⁵S]UTP (1250 Ci/mmol; NEN, Massachusetts, USA) in the presence of T7 or T3 RNA polymerase. The in situ hybridization was carried out on a mouse tissue fixed with formaldehyde and embedded in paraffin. Sections (4 μm wide) were then deparaffinized in toluene and rehydrated in an alcohol gradient. After drying, the various sections were incubated in a prehybridization buffer for two hours. The hybridization was carried out overnight in a hybridization buffer (prehybridization buffer with 10 mM DTT and 2×10⁶ cpm RNA/μl ³⁵S-labeled) at 53° C. The excess probe was removed and the sections were inclined in an LM1 emulsion (Amersham Biosciences, UK) and exposed in the dark at 4° C. for at least one month. The sections were then developed and counterstained with hematoxylin and eosin. The hybridization with the sense probe was used as negative control and only the background was detected. These probes were incubated with histological sections of mouse skin or mouse preputial gland. Following incubation in the presence of a photographic emulsion, the histological structures radioactively labeled with the probe are visualized (accumulation of silver grains). A specific signal manifests itself by a positive labeling with the anti-sense probe (FIG. 3B and FIG. 3C) and the absence of labeling with the sense probe (FIG. 3A) used as negative control.

Results:

It is observed in FIG. 3A that there is no accumulation of silver grains (no labeling), which is in agreement with the expectations of the inventors because it corresponds to the negative control. FIG. 3B shows a strong labeling of the basal layer of the sebaceous gland, visible by accumulation of silver grains. FIG. 3C also shows a labeling of the basal layer of the sebaceous gland.

LSS is expressed in the basal layers of the sebaceous glands of mouse skin. Fine analysis based on the observation of histological sections obtained for 4 intact animals and 4 gonadectomized animals shows a higher expression in the sebaceous glands of the intact animals than in the gonadectomized animals. This result is in agreement with the induction of the gene by an androgenic stimulation observed in the Affymetrix and RT-PCR experiments.

### Example 4

### Expression of LSS in the Mouse Preputial Gland by “In Situ Hybridization”

Methods:

The methods used in this example are identical to those of Example 3.

The mouse preputial glands show a sebocyte type differentiation and are used as an experimental model of the sebaceous gland.

Results:

FIG. 4A shows no labeling at the level of the preputial gland, which is in agreement with the expectations of the inventors because it corresponds to the negative control. FIG. 4B shows a very high labeling of the mouse preputial gland in a normal animal. FIG. 4C shows a more moderate labeling of the acini of the preputial gland in a gonadectomized animal.

LSS is expressed in the mouse preputial gland, in particular in the basal layers of the acini. Analysis of several histological sections from 4 control animals and 4 gonadectomized animals indicates a markedly higher expression in the preputial glands of the intact animals.

Briefly, the results of in situ hybridization in the mouse preputial gland indicate that the expression of the LSS enzyme increases under conditions characterized by an androgenic stimulation (intact animals). These observations are in agreement with the data obtained by the Affymetrix technologies and the real-time PCR.

### Example 5

### Examples of Compositions

### A—Oral Route

| | |
|---|---|
| Ro 48-8071 | 0.001 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

### B—Topical Route

(a) Salve

| | |
|---|---|
| Ro 48-8071 | 0.300 g |
| Petroleum jelly | qs 100 g |

(b) Lotion

| | |
|---|---|
| Ro 48-8071 | 0.100 g |
| Polyethylene glycol | 69.900 g |
| (PEG 400) | |
| Ethanol at 95% | 30.000 g |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. An in vitro method for screening candidate compounds for the preventive and/or curative treatment of acne, or skin disorders associated with a hyperseborrhea, comprising determining the capacity of a candidate compound to modulate the expression or activity of lanosterol synthetase (LSS) or the expression of its gene or the activity of at least one of its promoters.

2. An in vitro method for screening candidate compounds for the preventive and/or curative treatment of acne or skin disorders associated with a hyperseborrhea as defined by claim 1, comprising the following steps:
a. preparing at least two biological samples or reaction mixtures;
b. bringing one of the samples or reaction mixtures into contact with one or more test compounds;
c. measuring the expression or activity of the protein lanosterol synthetase, the expression of its gene or the activity of at least one of its promoters, in biological samples or reaction mixtures;
d. selecting the compounds for which a modulation of the expression or activity of the protein lanosterol synthetase, or a modulation of the expression of its gene or a modulation of the activity of at least one of its promoters, is measured in the sample or mixture treated in b), compared with the untreated sample or mixture.

3. The in vitro method as defined by claim 2, wherein the compounds selected in step d) inhibit the expression or the activity of the protein lanosterol synthetase, the expression of its gene or the activity of at least one of its promoters.

4. The in vitro method as defined by claim 2, wherein the biological samples are cells transfected with a reporter gene that is operably linked to all or part of the promoter of the gene encoding lanosterol synthetase, and in that step c) comprises measuring the expression of the said reporter gene.

5. The in vitro method as defined by claim 2, wherein the biological samples are cells expressing the gene encoding lanosterol synthetase, and in that step c) comprises measuring the expression of the said gene.

6. The in vitro method as defined by claim 4, in which the cells are sebocytes.

7. The in vitro method as defined by claim 5, in which the cells are cells transformed with a heterologous nucleic acid encoding lanosterol synthetase.

8. The in vitro method as defined by claim 2, in which the expression of the gene is determined by measuring the level of transcription of the said gene.

9. The in vitro method as defined by claim 2, in which the expression of the gene is determined by measuring the level of translation of the said gene.

10. The in vitro method as defined by claim 2, wherein step a) comprises preparing reaction mixtures each comprising an enzyme lanosterol synthetase and a substrate of the enzyme, and in that step c) comprises measuring the enzyme activity.

11. The in vitro method as defined by claim 10, in which the determination of the enzyme activity comprises the determination of the synthetase activity by extraction of the sterols produced and chromatographic analysis.
